(19) United States
(12) Patent Application Publication
(10) Pub. No.: US 2006/0165637 A1
(43) Pub. Date: Jul. 27, 2006

(54) Topical agent containing phytanic acid or a derivative thereof

(76) Inventors: Jurgen Herber Vollhardt, Ramlinsburg (CH); Raphael Beumer, Lorrach (DE)

Correspondence Address: NIXON & VANDERHYE, PC, 901 NORTH GLEBE ROAD, 11TH FLOOR, ARLINGTON, VA, 22203, US

(21) Appl. No.: 10/559,625

(22) PCT Filed: Jun. 17, 2004
(86) PCT No.: PCT/EP04/06520

§ 371 Date: Dec. 6, 2005

(30) Foreign Application Priority Data
Jun. 17, 2003 (EP) 030137244

(51) Int. Cl. A61K 8/64; A61K 8/40

(52) U.S. Cl. 424/70.14; 424/70.27; 424/70.31

(57) ABSTRACT

The invention relates to agents for topical administration of a compound of formula (I): (CH₃)₂CH—(CH₂)₃—CH(CH₃)—(CH₂)₃—CH(CH₃)—(CH₂)₃—C(A)(CH₃)—C(B)₂—C(O)—R, wherein R represents hydrogen, OR¹, N(OH)R¹ or NR²R³; R¹, R² and R³ independently represent hydrogen C₁-C₂₂-alkyl, C₁-C₂₂-alkenyl, C₇-C₁₂-arylalkyl (particularly benzyl, phenethyl and phenylpropyl), retinyl, tocopheryl, ascorbyl or a radical stemming from an amino acid or a peptide and A and B represent hydrogen atoms or A and a radical B form a double bond and the other radical B represents a hydrogen atom or radical A represents a hydrogen atom and the radicals B together form an oxygen atom or one of the radicals B represents a hydroxyl group and the other radical B and radical A represent hydrogen atoms. Said agents contain a compound of formula (I) and a pharmaceutically and/or cosmetically acceptable carrier provided that the agent does not contain any retinoids.

The present invention relates to preparations for topical administration containing phytanic acid or a derivative thereof. The preparations are particularly suited for treating cellulite and/or subcutaneous fat pads but also for treating skin aging or a disturbed or dysfunctional epidermal barrier.

Orange skin or cellulite is a wide-spread esthetic problem from which many women suffer. Cellulite, also referred to as local lipodystrophy, initially develops as a result of changes in the lymph and blood circulation, which, in turn, causes structural rearrangements in the subcutaneous fat tissue and the surrounding collagen matrix. On account of these processes, the fat cells are separated from nutrition and excretion pathways and swell so as to form nodules in the millimeter range which are surrounded by solid collagen tissue. These nodules subsequently combine to form larger aggregates having diameters of up to 20 mm and force their way into the dermis. On account of the surrounding collagen fibers which remain anchored in the subcutis, constrictions occur which are considered the cause of the esthetic problem. In the late phase there may also be a special feeling of pain since the nodules irritate the nerve endings by pressure.

Cellulite is usually considered a cosmetic problem, however, cellulite can also be regarded as a disease requiring therapy. This is based on the fact that in particular in the region of the lymph vessels highly adipose subcutaneous tissues may also cause pains which require medicinal treatment. Cellulite may also be a heavy mental burden for persons suffering therefrom and call for a corresponding treatment.

Basically, the state of the art distinguishes between two methods to prevent and treat cellulite. On the one hand, mechanical treatments, such as massages, are used and, on the other hand, certain formulations applied to the skin. Those applied to the skin can be divided into three groups. The first group comprises preparations having active substances suited to promote restructuring of the protein network. They include e.g. retinoids (A. Kligman et al., Topical retinol improves cellulite, J. Dermatol. Treat. 10, 119-126, 1999, and J. Invest. Dermatol. 96, 975, 1991, Topical all-trans retinoic acid stimulates collagen synthesis). However, these preparations are not suited to positively influence the size of the fat pads, and retinoids are not well tolerated by many patients when applied topically.

A second group of formulations contains active substances which improve the blood supply. Here, above all formulations with caffeine are known. These preparations are above all effective in the early stage of cellulite as long as the blood vessels sufficiently penetrate the subcutaneous adipose tissue. However, as the condition proceeds, the number of blood vessels decreases drastically in the swollen adipose tissue and such preparations loose effectiveness.

A third group of methods try to positively influence the lipometabolism. The balance between lipolysis and lipogenesis determines the size of the fat pads and is thus an essential factor which, in the case of an unbalance for the benefit of lipogenesis, assists in the development of cellulite. For example, WO 03/009826 describes the use of steroids to restore this balance. However, the use of steroids may cause considerable side-effects.

A number of preparations usable against skin aging or in the case of a disturbed barrier function of the epidermis (a disturbed or injured skin barrier) are known, e.g. from WO 01/43704 or WO 98/32444.

WO 01/43704 discloses a number of compounds which can be applied topically to the skin and which support the biosynthesis and/or bioactivity of endogenous chemicals. In particular, the compounds shall procure the communication between keratinocytes, fibroblasts and other cell types of the skin by activating the gene expression improving the cellular activity. WO 01/43704 mentions a number of compounds having such an activity, among them being phytol and derivatives thereof. This publication does not relate to either phytanic acid or the treatment of cellulite and subcutaneous fat pads.

WO 98/32444 relates to a method of treating the epidermis in a patient who has a disturbed barrier function of the epidermis and proposes as active substance an activator for certain receptors. Neither phytanic acid nor derivatives thereof are disclosed in this publication, and the treatment of cellulite and/or subcutaneous fat pads is not the subject matter of this WO publication either.

WO 01/64177 describes the use of flavones or isoflavones for treating cellulite.

DE 199 40 415 describes that natural fatty acids of the isoprenid and acetogenin type having methyl and ethyl branches and synthetic branched-chain fatty acids as health food products and additives to foodstuffs and semi-luxuries may promote the lipid catabolism in man. This publication relates exclusively to the systemic absorption of the active substances, and the invention described therein is based on the fact that the active substances interact with binding sites which are far from the target of treatment, i.e. the skin. A topical application is not described in this publication. Medicaments requiring systemic absorption are usually fully unsuited for the topical treatment of skin conditions. On the one hand, the systemic absorption results in the preferred interaction of the active substance with binding sites far from the target of treatment and unselective interactions also cause undesired side-effects; on the other hand, active substances metabolize in the skin in a completely different way since in the skin enzyme systems are active unlike those in the liver, for example.

WO 01/66080 discloses that phytol may support the effect of retinoids to improve the condition of the skin. In particular, the phytol-supported retinoids shall counteract the impact of aging processes in epidermis and dermis. This publication assumes that phytol is converted into phytanic acid after being applied to the skin. Indeed, this conversion can take place to a certain extent in the liver following oral uptake. However, recent studies have shown that this conversion does not occur when phytol is applied topically to the skin. Therefore, when applied topically to the skin phytol is no precursor medicament (prodrug) of phytanic acid contrary to the disclosure of WO 01/66080.

Phytanic acid is known to be a potential RXR agonist, as described in WO 01/66080. However, the binding affinity is 200 times weaker than that of retinoic acid, and therefore cosmetic effects, such as the anti-wrinkle effect known for retinoic acid, cannot be expected of phytanic acid already because of the very weak RXR binding. When fighting for the RXR binding site, phytanic acid would have no chance compared with the naturally present Ligand, i.e. retinoic acid.

WO 01/66080 does not relate to the treatment of cellulite either and does not disclose a retinoid-independent phytol effectiveness of its own in the case of topical application. However, undesired side-effects can occur when skin diseases are treated with retinoids.

Therefore, there is a demand for new preparations which can be used both cosmetically and pharmaceutically and which following topical application are particularly effective for treating cellulite and/or subcutaneous fat pads. The preparations shall also slow down or preferably reverse skin aging, i.e. in particular smooth skin wrinkles and little crinkles, reduce age spots and improve the mechanical properties of the skin, such as smoothness, texture, elasticity, and improve the skin tone and the uniform color. It is preferred for the preparations according to the invention to also repair as quickly as possible an affected or damaged skin barrier thus improving the skin moisture, i.e. treat in particular also dry skin or prevent the occurrence of dry skin.

It is the object of this invention to provide such a preparation which in addition has the least possible side-effects and does not have the drawbacks of the known preparations of the prior art.

This object is achieved on the basis of the surprising finding that phytanic acid and derivatives thereof of formula 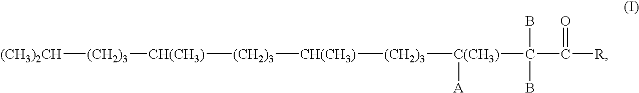
wherein R is hydrogen, OR¹, N(OH)R¹ or NR²R³,
R¹, R² and R³ are independently hydrogen, C₁-C₂₂ alkyl, C₁-C₂₂ alkenyl, C₇-C₁₂ arylalkyl (in particular benzyl, phenethyl and phenylpropyl), retinyl, tocopheryl, ascorbyl or a residue derived from an amino acid or peptide, and A and B are hydrogen atoms or A and a residue B form a double bond and the other residue B is a hydrogen atom or residue A is a hydrogen atom and residues B are together an oxygen atom or one of residues B is a hydroxyl group and the other residue B and residue A are hydrogen atoms, can prevent cellulite and/or subcutaneous fat pads when applied topically to the skin and are also effective for treating cellulite and/or subcutaneous fat pads as well as have excellent properties in the cosmetic and medicinal treatment of skin conditions as defined below.

In a specific embodiment one of residues R² and R³ is a hydrogen atom and the second residue is as defined above.

In a further preferred embodiment one of residues B is a hydroxyl group.

In a further preferred embodiment both residues B form an oxygen atom.

The arylalkyl residues are bound via the alkyl unit.

In a particularly preferred embodiment, the invention relates to the use of compounds of formula 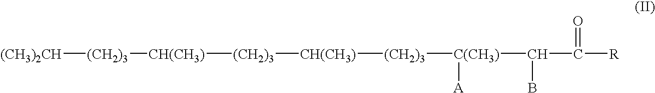
wherein R is hydrogen, OR¹, NHR¹ or N(OH)R¹,
R¹ is hydrogen, C₁-C₂₂ alkyl, C₁-C₂₂ alkenyl, benzyl, phenethyl, phenpropyl, retinyl, tocopheryl, ascorbyl or a residue derived from an amino acid or a peptide, and A and B are either both hydrogen atoms or together form a double bond.

In a specific embodiment of the compounds of formulae I and II, R is hydrogen, OR¹, NHR¹ or N(OH)R¹ and R¹ represents hydrogen, C₁-C₂₂ alkyl, C₁-C₂₂ alkenyl, benzyl, phenethyl, phenpropyl, retinyl, tocopheryl, ascorbyl or a residue derived from an amino acid or a peptide, and A and B are either both hydrogen atoms or together form a double bond.

The preparations according to the invention are cosmetic preparations where the phytanic acid or the derivative thereof is formulated with cosmetically compatible additives and also pharmaceutical preparations where the phytanic acid or the derivative thereof is formulated with pharmaceutically compatible additives. Unless otherwise stated in this application, disclosed additives are both cosmetically compatible additives and pharmaceutically compatible additives.

The invention also provides the use of phytanic acid and derivatives thereof as defined above to produce topical cosmetic preparations and to produce topical preparations for preventing and/or treating cellulite, subcutaneous fat pads, skin aging, in particular smoothing of skin wrinkles and small crinkles, reduction of age spots, improvement of the mechanical properties of the skin, such as smoothness, texture, elasticity, improvement of the skin tone and the uniformity of color as well as to treat or repair a damaged or injured skin barrier.

The invention also provides the cosmetic use of phytanic acid or a derivative thereof as defined above to treat cellulite, subcutaneous fat pads, skin aging, in particular smoothing of skin wrinkles and small crinkles, reduction of age spots, improvement of the mechanical properties of the skin, such as smoothness, texture, elasticity, improvement of the skin tone and the uniformity of color as well as to treat and/or repair a damaged or injured skin barrier.

The use of phytanic acid or a derivative thereof as defined above is useful e.g. for treating and actively preventing dry skin and strengthening the barrier function of the skin and for treating, caring for and preventing sensitive skin and/or for treating and preventing the symptoms or a negative change in the physiological homeostasis of the healthy skin, in particular inadequate, sensitive or hypoactive skin conditions or inadequate, sensitive or hypoactive conditions of skin appendages, inflammatory skin conditions as well as the atopic eczema, the polymorphous photodermatosis, psoriasis, vitiligo, sensitive, itching or irritated skin, changes in the normal lipid peroxidation, a change in the ceramide, lipid and energy metabolism of the healthy skin, a change in the physiological transepidermal loss of water, a reduction of the skin hydration and decrease of the moisture content of the skin, a change in the natural moisturizing factor content, reduction of the cell-to-cell communication, deficiency symptoms of the intracellular DNA synthesis, DNA damage and reduction of the endogenous DNA repair mechanisms, activation of metalloproteinases and/or other proteases or inhibition of the corresponding endogenous DNA repair mechanisms and deviations from the normal post-translational modifications of connective tissue components.

Phytanic acid regulates the sebum production of the skin and prevents an excessively strong production of sebum. In the region of the scalp, regreasing of the hair is reduced following washing. Phytanic acid-containing hair care products are thus very well suited for easily greasing hair or for short hairstyles where regressing can be seen very rapidly. The regulation of the skin lipids also has an advantageous effect on the scalp since it counteracts the formation of dandruff. The formation of dandruff is supported in particular by dry skin. According to the invention phytanic acid can therefore also be used for treating and preventing dandruff.

As a result, the invention also relates to the use of phytanic acid or a derivative thereof as defined above as a hair care product which is permanent or can be washed out, such as deep conditioners or shampoos, in particular to the treatment and/or prevention of greasy hair and/or dandruff formation.

According to the invention phytanic acid and the derivative thereof are not used together with a retinoid, and phytanic acid does not serve for enhancing another ingredient, such as a retinoid. The preparations according to the invention preferably contain no retinoid. According to the invention it is also preferred to use phytanic acid or the derivative thereof as the sole active substance for treating cellulite and/or subcutaneous fat pads.

According to the invention, it is also preferred to use phytanic acid with one or more further active substances, selected from

caffeine

flavones and isoflavones, e.g. genistein

carnitine

aescine

steroids, such as those mentioned in WO 03/009826

ruscogenin

dexpanthenol, panthenol,

nicotinates, such as vitamin E nicotinate and benzyl nicotinate

niacinamide

vitamins, ascorbyl glycosides or sodium ascorbyl phosphate

menthol

salicylic acid

disodium rutinyl disulfate

phloridzine

coenzyme A

hesperidine methyl chalcon

methyl silanol mannuronate

plant extracts, such as:

algae extracts, such as fucus vesiculosus extract, green tea or mate tea extract, Centella asiatica extract,

Hedera helix, Hieracium pilosella, Malva sylvestris, Panax ginseng

Citrus aurantium amara (bitter organe) flower extract,

apple extract (pyrus malus), guarana (paullinia cupana) extract

cola extract, horse chestnut extract (Aesculus hippocastanum extract),

ginkgo biloba.

In so far as a “preparation” is mentioned in this description without a more detailed specification following, this is a cosmetic preparation as well as a medicament. In order to distinguish between cosmetic preparations and medicaments reference can be made to Römpp, Chemielexikon, 10^{th} edition and literature cited therein, for example.

As to a definition of the term “retinoid” reference is made to WO 01/66080. The expression “retinoid” is defined in the present application in a way the same as that of WO 01/66080.

The term “phytanic acid” as used in this description refers to 3,7,11,15-tetramethylhexadecanoic acid. Of course, the acid occurs in two forms, i.e. in the 3R,7R,11R form and in the 3S,7R,11R form. According to the invention the term “phytanic acid” refers to any naturally occurring form separately or in admixture as well as to other forms of phytanic acid and to mixtures of one or more non-naturally occurring forms of phytanic acid, optionally in admixture with one or both naturally occurring forms. According to the invention phytanic acid is preferably used in a naturally occurring form or in admixture of both naturally occurring forms. Phytanic acid is a known compound and commercially available. All epimers of phytanic acid are comprised.

Along with the use of phytanic acid it is also preferred according to the invention to use a derivative of phytanic acid as defined above, in particular a derivative which is fully or partially converted into phytanic acid on or in the skin or before or during the application. It is particularly preferred for the phytanic acid derivatives used according to the invention to be phytanic acid esters, in particular alkyl ester, e.g. C₁-C₁₀ alkyl ester of phytanic acid. C₁-C₆ alkyl esters, in particular the methyl esters, ethyl esters, iso-propyl esters, n-propyl esters, n-butyl esters and tert-butyl esters can be mentioned as being particularly preferred. The esters of phytanic acid may be obtained from phytanic acid in known manner according to standard methods. Suitable methods of producing the preferred esters of phytanic acid are described in the examples.

Also preferred according to the invention are compounds of formula (I) as defined above, wherein residue R¹ is a C₁-C₁₀ alkenyl residue, in particular a C₁-C₆ alkenyl residue. The alkenyl residue preferably has less than three double bonds, in particular one or two double bonds. Also particularly preferred are compounds of formula (I) as defined above, wherein residue R¹ represents an ascorbyl residue. Residue R¹ can also be an amino acid or peptide residue. Such a compound is a typical proform for phytanic acid since it represents a substrate for proteases or amidases converting the compound into phytanic acid. If residue R is a hydrogen atom, the compound of formula (I) is phytal and according to the invention all epimers of phytal are also comprised. If residues A and B together form double bonds which are phytenic acid derivatives, and of these compounds all epimers and both the E form and the Z form are comprised. However, A and B preferably represent hydrogen atoms and, if A and B together form a double bond, residue R¹ is preferably a hydroxyl group so that the compound then represents phytenic acid per se. The compounds may be produced in known manner using chemical standard methods, based on phytanic acid, for example.

Compounds of formula II as defined above, wherein residue R represents OR¹ and R¹ is an n-propyl or C₄-C₂₂ alkyl residue are also novel, and the invention also relates to these novel compounds as such.

The derivatives of phytanic acid are either active against cellulite and/or subcutaneous fat pads per se or are converted into the active phytanic acid before, during or after the topical application.

The preparations according to the invention are suited in particular for the cosmetic or medicinal treatment of cellulite or subcutaneous fat pads. Moreover, they are markedly effective for preventing and treating skin aging, in particular for smoothing skin wrinkles and small crinkles, for reducing age spots, for improving mechanical properties of the skin, such as smoothness, texture and elasticity, and furthermore they improve the skin tone and the uniformity of the color. The preparations according to the invention are also of special benefit for the cosmetic or medicinal treatment of a disturbed or injured skin barrier (epidermal barrier) and conditions evoked by this, as mentioned in WO 98/32444, for example, in particular fluid or electrolyte anomalies, hypothermia and infections through the skin of premature children younger than 33 weeks, inflammations of the mucous membranes, such as chilitis, chapped lips, nasal irritations and vulvovaginitis, eczematous dermatitis, such as atopic or seborrheic dermatitis, allergic dermatitis or non-allergic contact dermatitis, cracked eczema, photoallergic dermatitis, phototoxic dermatitis, phytophotodermatitis, radiation dermatitis and statis dermatitis, ulcera and superficial skin defects, caused by trauma, burns, bullous diseases or skin or mucosa ischemia, several forms of ichthyosis, epidermolysis bullosa, psoriasis, hypertrophic scars and cheloids, dermal changes of intrinsic aging of photoaging, blister formation caused by friction as a result of mechanical shearing of the skin and dermal atrophy on account of the topical use of corticosteroids.

With respect to the diseases to be treated reference is made to the full content of WO 98/32444.

The preparations according to the invention contain phytanic acid and/or a derivative of phytanic acid and suitable cosmetic and/or pharmaceutically compatible additives.

It is particularly preferred that the active substance, i.e. phytanic acid or the derivative of phytanic acid, is contained in the preparations according to the invention in an amount of 0.0001% by weight to about 50% by weight, based on the total weight of the composition. More preferably, phytanic acid or the derivative of phytanic acid is contained in an amount of 0.01% by weight to about 20% by weight, even more preferably in an amount of about 0.1% by weight to about 15% by weight, e.g. 1 to about 5%, based on the total weight of the composition.

The preparations according to the invention comprise one or more cosmetically compatible or pharmaceutical compatible carriers and/or additives or active substances as usually used in such preparations. Here, fats, oils, waxes, silicones, emulsifiers, alcohols, polyols, thickening agents, moistening and/or moist-keeping substances, surfactants, plasticizers, foam suppressants, anionic, cationic, non-ionic or amphoteric polymers, alkanization or acidification agents, softening agents, adsorbents, light-stability agents, electrolytes, masking agents, organic solvents, preservatives, bactericides, antioxidants, vitamins, aromatic principles, aromas, sweeteners, dyes and pigments can be mentioned by way of example.

Suitable compositions are e.g. liquid or solid oil-in-water emulsions, water-in-oil emulsions, multiple emulsions, microemulsions, PIT emulsions, pickering emulsions, hydrogels, alcoholic gels, lipogels, single-phase or multi-phase solutions, foams, ointments, plasters, suspensions, powders, creams or other conventional preparations. The preparations according to the invention can also be formulated in an anhydrous form, such as oil or balm, e.g. with vegetable or animal oils, mineral oils, synthetic oils or mixtures thereof as carrier substances.

Suitable formulations for treating cellulite are described in WO 01/64177 for the active substances flavones and isoflavones. The topical formulations described therein for treating cellulite are in principle also suited for formulating phytanic acid and the derivatives thereof, the active substance or the plant extract from the formulations of WO 01/64177 being replaced by phytanic acid or a derivative thereof. In this respect, reference is made to the disclosure of WO 01/64177.

The formulations according to the invention preferably contain one or more conventional fatty substances, e.g. vegetable oils, liquid paraffin oils, isoparaffin oils, synthetic hydrocarbons, di-n-alkyl esters, fatty acids, fatty alcohols, ester oils, hydroxycarbonic acid alkyl esters, dicarbonic esters, diol esters, symmetric, asymmetric or cyclic esters or carbonic acid with fatty alcohols, mono-, di- and trifatty acid esters of saturated and/or unsaturated linear and/or branched fatty acids with glycerol, waxes and silicone compounds. Suitable examples of such fatty substances are disclosed in WO 01/64177 to which reference is made in this respect.

The fatty substances are usually present in an amount of 0.1 to 50% by weight, preferably 0.1 to 20% by weight, in particular 0.1 to 15% by weight, in the preparation according to the invention (each based on the entire preparation).

Like the preparations of WO 01/64177, the preparations according to the invention may contain one or more surface-active substances as emulsifiers or dispersing agents. Suitable examples of such emulsifiers or dispersing agents are mentioned in WO 01/64177 to which reference is made in this respect.

The emulsifiers may be contained in the preparations according to the invention in proportions of e.g. 0.1 to 25% by weight, more preferably 0.5 to 15% by weight, based on the entire preparation.

The preparations according to the invention may also contain conventional light-stability agents, e.g. conventional UV-A and/or UV-B filters. A list of conventional UV-A and UV-B filters, which may also be used in the preparations according to the invention, is found in EP-A-1 081 140, for example. According to the invention the novel dark filters disclosed for the first time in this publication can, of course, also be used in the preparations according to the invention.

Suitable organic, mineral or modified mineral light-stability filters are also mentioned in WO 01/64177 to which reference is made in this respect.

If desired, the preparations according to the invention can also contain protein hydrolysates or derivatives thereof as well as suitable mono-, oligo- or polysaccharides or the derivatives thereof, as disclosed in WO 01/64177. Further suitable auxiliary substances and additives, such as vitamins, provitamins and vitamin precursors, alantopine, bisabolol, antioxidants, ceramides and pseudoceramides, triterpenes, monomeric catechines, thickeners, vegetable glycosides, structure-imparting agents (structurants), dimethyl isosorbide, solvents, swelling and penetration aids, perfume oils, pigments and dyes for staining the preparation, substances for adjusting the pH, complexing agents, opacifiers, pearlescent agents, blowing agents, film-forming, emulsion-stabilizing, thickening or adhesive polymers, in particular cationic, anionic and non-ionic polymers are also disclosed in WO 01/64177 to which reference is made in this respect.

The preparations according to the invention are formulated as usual. The below examples exemplify the production of an O/W emulsion. The production of these formulations and others is known to the person skilled in the art, and reference can be made here to conventional formulation textbooks.

The preparations according to the invention are formulated such that they are suited for topical administration. The topical administration is made at least once a day, e.g. twice or three times a day. The treatment period is usually at least two days until the desired effect has been achieved. The treatment period can also be several weeks or months. The treatment of a damaged skin barrier can already be concluded successfully after a relatively short period of time, such as 1 day to 1 week, whereas the treatment of cellulite and skin aging usually takes 1 to 2 months.

The amount of preparation to be applied depends on the concentration of the active substance in the preparation and the severity of the disease to be treated or the desired cosmetic result. As a rule, the active substance amount to be applied per application for pharmaceutical use is greater than for cosmetic use. A suitable amount for the application depends on the quality of the skin, the person to be treated and the severity of the cellulite to be treated and other factors, which are known to an attending physician or cosmetician. For example, the application may be such that a cream is applied onto the skin. A cream is usually applied in an amount of 2 mg cream/cm² skin. For treating cellulite or the subcutaneous fat pads, an amount of active substance used should be about 10 μg to 1 mg/cm² skin. A cream for treating cellulite or the subcutaneous fat pads should thus contain 0.5% by weight to 50% by weight of active substance of formula I. For a repair of the barrier function of the skin, often smaller active substance amounts, such as 2 μg/cm² skin, suffice so that the topical preparations may have a correspondingly smaller active substance concentration. However, the amount applied is not critical, and if a certain applied active substance amount does not yield a treatment success, the applied amount can readily be raised, e.g. by using topical formulations having higher concentrations.

The preparations according to the invention usually contain 0.05 to 50% by weight of formula I, more preferably 0.1 to 40% by weight, e.g. 0.5 to 40% by weight. The active substance concentration is preferably chosen such that when a common amount of the preparation is applied to the skin, 1 μg to 2 mg active substance/cm² skin, more preferably 2 μg active substance to 1 mg active substance/cm² skin, e.g. 10 μg to 500 μg active substance/cm², are provided.

A special advantage of the formulations according to the invention is that they can particularly rapidly restore the healthy condition of the skin. In particular, body lotions are of benefit which are applied after washing the skin, which is accompanied by a destruction of the skin barrier by dissolving out barrier lipids. This problem is also described expressly in WO 98/32444, for example.

According to the invention, the active substances of formula I can be used as such or also in liposomal form. Liposomes are advantageously formed with lecithins without or with the addition of sterols or phytosterols. The active substances of formula I can be encapsulated as such or together with other active substances.

The preparations according to the invention are particularly suited for treating humans but can also be used for treating animals.

The following examples explain the invention.

### EXAMPLE 1

Production of Phytanic Acid Ethyl Ester

### 3,7,11,15-Tetramethylhexadecanoic acid ethyl ester

3,7,11,15-tetramethylhexadecanoic acid (28.9 g, 90.0 mmol) is dissolved in dried CHCl₃ (100 ml). Ethanol is added in excess (157.5 mmol) together with concentrated H₂SO₄ (450 mg), and the solution is refluxed on a Soxhlet apparatus with a molecular screen (4 Å) for four days. The reaction mixture is then washed out in a separating funnel with aqueous 10% sodium hydrogen carbonate solution (2×100 ml). The combined aqueous phases are extracted once again with ethyl acetate (2×100 ml). The combined organic phases are then dried on sodium sulfate, and the solvent is distilled off under reduced pressure. The residue is distilled at 140° C., 3.0×10⁻¹ mbar. Yield: 28.6 g pure substance, 93% yield. −R_{f} (n-hexane/ethyl acetate 9:1)=0.78; ¹H-NMR (400 MHz, CDCl₃): δ=4.12 (q, J=7.2, 2H), 2.33-2.27 (m, 1H), 2.15-2.06 (m, 1H), 2.02-1.89 (m, 1H), 1.59-1.46 (m, 1H), 1.44-1.02 (m, 24H), 0.98-0.80 (m, 14H); MS (EI): 340 (12) [M⁺], 115 (100) [C₈H₁₁O₂⁺]; IR (film) cm⁻¹: ν=2925, 1737, 1462, 1376, 1165, 1033, 930, 847.

### EXAMPLE 2

Production of Phytanic Acid n-butyl Ester

### 3,7,11,15-Tetramethylhexadecanoic acid n-butyl ester

3,7,11,15-Tetramethylhexadecanoic acid (10.0 g, 32.0 mmol) is dissolved in n-butanol in excess (160.0 mmol). Then, concentrated sulfuric acid (345 mg) is added, and the solution is refluxed on a soxhlet apparatus with a molecular screen (4 Å) for four days. The reaction mixture is then washed out in a separating funnel with aqueous 10% sodium hydrogenate carbonate solution (2×100 ml). The combined aqueous phases are extracted once again with ethyl acetate (2×100 ml). The combined organic phases are then dried on sodium sulfate, and the solvent is distilled off under reduced pressure. The residue is distilled at 142° C., 4.4×10⁻¹ mbar. Yield: 9.8 g pure substance, 83% yield. −R_{f} (n-hexane/ethyl acetate 49:1)=0.48; ¹H-NMR (400 MHz, CDCl₃): δ=4.07 (t, J=6.8 Hz, 2H), 2.32-2.26 (m, 1H), 2.18-2.06 (m, 1H), 1.99-1.90 (m, 1H), 1.66-1.57 (m, 2H), 1.55-1.46 (m, 1H), 1.43-1.01 (m, 22H), 0.97-0.90 (m, 6H), 0.88-0.82 (m, 12H); MS (EI): 368 (20) [M⁺], 143 (100) [C₈H₁₅O₂⁺]: IR (film) cm⁻¹: ν=2925, 2869, 1736, 1462, 1378, 1166, 1022.

The following formulation examples are % by weight indications based on the entire weight of the composition.

### FORMULATION EXAMPLE 1

Anti-cellulite cream with caffeine

| | components | % by weight |
|---|---|---|
| A) | Arachidyl Alcohol & Behenyl Alcohol & | 5.00 |
| | Arachidyl Glucoside | |
| | Isononyl Isononanoate | 4.00 |
| | Mineral Oil | 4.00 |
| | Dow Corning Silicone 345 | 2.00 |
| | (Cyclomethicone) | |
| | Cetyl Alcohol | 2.00 |
| | Phytanic acid ethyl ester | 1.00 |
| | Squalane | 2.00 |
| | Dow Corning Silicone DC 200/100 | 0.50 |
| | (Dimethicone) | |
| | BHT | 0.05 |
| | Phenonip (Phenoxyethanol & Parabens) | 1.00 |
| B) | Aqua | q.s. |
| | | (based on components |
| | | A, B and C) |
| | Caffeine | 5.00 |
| | Glycerol | 4.00 |
| | Butylene Glycol | 2.00 |
| | Acrylates/C10-30 Alkyl Acrylate | 0.20 |
| | Crosspolymer | |
| | Disodium EDTA | 0.10 |
| C) | Panthenol | 1.00 |
| | Tocopherol Acetate | 0.50 |
| | Fragrance | 0.10 |
| D) | Triethanolamine | q.s. |

Manufacturing Instruction

Heat parts A and B separately to 80° C. Slowly add part A to part B with vigorous stirring using the Ultraturax at 13000 rpm and homogenize for two minutes. Allow the emulsion to cool down to 45° C. and add the ingredients of part C with slow stirring. Then, use part D to adjust the pH to 6.0.

### FORMULATION EXAMPLE 2

Anti-cellulite cream

| | Components | % by weight |
|---|---|---|
| A) | Arachidyl Alcohol & Behenyl Alcohol & | 5.00 |
| | Arachidyl Glucoside | |
| | Isononyl Isononanoate | 2.00 |
| | Mineral Oil | 4.00 |
| | Dow Corning Silicone 345 | 2.00 |
| | (Cyclomethicone) | |
| | Cetyl Alcohol | 2.00 |
| | Squalane | 1.00 |
| | Phytanic acid n-butyl ester | 4.00 |
| | Dow Corning Silicone DC 200/100 | 0.50 |
| | (Dimethicone) | |
| | BHT | 0.05 |
| | Phenonip (Phenoxyethanol & Parabens) | 1.00 |
| B) | Aqua | q.s. |
| | | (based on components |
| | | A, B and C) |
| | Glycerol | 4.00 |
| | Butylene Glycol | 2.00 |
| | Acrylates/C10-30 Alkyl Acrylate | 0.20 |
| | Crosspolymer | |
| | Disodium EDTA | 0.10 |
| C) | Panthenol | 1.00 |
| | Tocopherol Acetate | 0.50 |
| | Fragrance | 0.10 |
| D) | Triethanolamine | q.s. |

Manufacturing Instructions

Heat parts A and B separately to 80° C. Add slowly part A to part B with vigorous stirring using the Ultraturax at 13000 rpm and homogenize for two minutes. Allow the emulsion to cool down to 45° C. and add the additives of part C with slow stirring. Thereafter, use part D to adjust the pH to 6.0.

### FORMULATION EXAMPLE 3

Anti-cellulite cream

| | Components | % by weight |
|---|---|---|
| A) | Phytanic acid | 2.00 |
| | Glyceryl Stearate SE | 5.00 |
| | 2-Octyldodecanol | .00 |
| | Mineral Oil | 4.00 |
| | Dow Corning Silicone 345 | 2.00 |
| | (Cyclomethicone) | |
| | Cetaryl Alcohol | 2.00 |
| | Stearic acid | 1.00 |
| | Squalane | 2.00 |
| | Dow Corning Silicone DC 200/100 | 0.50 |
| | (Dimethicone) | |
| | Phenonip (Phenoxyethanol & Parabens) | 0.50 |
| B) | Aqua | q.s. |
| | | (based on components |
| | | A, B and C) |
| | Caffeine | 1.00 |
| | Glycerol | 4.00 |
| | Carnosine | 0.20 |
| | Genistein | 0.10 |
| | Acrylates/C10-30 Alkyl Acrylate | 0.20 |
| | Crosspolymer | |
| | Disodium EDTA | 0.10 |
| C) | Panthenol | 1.00 |
| | Tocopherol Acetate | 0.50 |
| | Fragrance | 0.20 |
| D) | Potassium hydroxyde | q.s. |

Manufacturing Instructions

Heat parts A and B separately to 80° C. Add slowly part A to part B with vigorous stirring using the Ultraturax at 13000 rpm and homogenize for two minutes. Allow the emulsion to cool down to 45° C. and add the additives of part C with slow stirring. Thereafter, use part D to adjust the pH to 7.5.

### FORMULATION EXAMPLE 4

Slimming Lotion using phytanic acid n-butyl ester

| | components | % by weight |
|---|---|---|
| A) | Glyceryl Myristate | 4.00 |
| | Cetyl Alcohol | 1.00 |
| | Ethylhexyldodecanol | 2.00 |
| | Phytanic acid n-butyl ester | 3.00 |
| | Dimethicone | 2.00 |
| | Tocopheryl acetate | 2.00 |
| | Disodium EDTA | 0.10 |
| | Phenoxyethanol & Methylparaben &Ethylparaben & | 0.60 |
| | Propylparaben & Butylparaben | |
| | Cetyl Phosphate | 0.84 |
| B) | Aqua | 10.00 |
| | Potassium Hydroxide | 1.60 |
| C) | Aqua | ad 100 |
| | Carbomer | 0.10 |
| | Propylene Glycol | 5.00 |
| D) | Potassium Hydroxide | 0.50 |
| E) | Sodium Ascorbyl Phosphate | 0.50 |
| | Aqua | 10.00 |

Manufacturing Instructions

Heat part A to 85° C. with stirring. As soon as everything is dissolved, add part B. Then, slowly introduce part C heated to 80° C. with vigorous stirring using the Ultraturax at 13000 rpm. Also slowly add part D. Homogenize for one minute. Allow the emulsion to cool down to 40° C. and add the additives of part E with slow stirring. Thereafter, adjust the pH to 6.0 using the potassium hydroxide solution.

| | Formulation | |
|---|---|---|
| | examples | |
| | 5 | 6 |
| | % | % |
| Ingredients | (w/w) | (w/w) |
| Glyceryl Myristate | 4.00 | 4.00 |
| Cetyl Alcohol | 2.00 | 2.00 |
| Steareth-2 | 2.00 | 2.00 |
| Steareth-21 | 2.00 | 2.00 |
| Isopropyl Myristate | 5.00 | 5.00 |
| Tocopheryl Acetate | 0.50 | 0.50 |
| Almond oil | 2.00 | 2.00 |
| BHT | 0.05 | 0.05 |
| Phenoxyethanol & Methylparaben & Ethylparaben & | 0.80 | 0.80 |
| Propylparaben & Butylparaben & Isopropylparaben | | |
| Aqua | Ad. 100 | Ad. 100 |
| Dinsodium EDTA | 0.10 | 0.10 |
| D-Panthenol | 0.30 | 0.30 |
| Sodium ascorbyl phosphate | 0.50 | 0.50 |
| Propylene glycol | 4.00 | 4.00 |
| Polyacrylamide & C13-14 Isoparaffin & Laureth-7 | 0.50 | 0.50 |
| Phytanic acid | 0.50 | 1.00 |
| Triethanolamine | q.s. | q.s. |
| | Formulation | |
| | examples | |
| | 7 | 8 |
| | % | % |
| Ingredients | (w/w) | (w/w) |
| Aqua | Ad. 100 | Ad. 100 |
| Acrylate/C10-30 Alkylacrylate Crosspolymer | 0.60 | 0.60 |
| NaOH 30% | 0.40 | 0.40 |
| Disodium EDTA | 0.10 | 0.10 |
| D-Panthenol | 0.50 | 0.50 |
| Squalane | 2.00 | 2.00 |
| Coco-Carylat/Caprat | 4.00 | 4.00 |
| BHT | 0.05 | 0.05 |
| Phenoxyethanol & Methylparaben & Ethylparaben & | 0.80 | 0.80 |
| Propylparaben & Butylparaben & Isopropylparaben | | |
| Cyclomethicon | 4.00 | 4.00 |
| Glycerol | 3.00 | 3.00 |
| Tocopherylacetate | 0.30 | 0.30 |
| Phytanic acid | 0.50 | 1.00 |
| | Formulation | |
| | examples | |
| | 9 | 10 |
| | % | % |
| Ingredients | (w/w) | (w/w) |
| Aqua | Ad. 100 | Ad. 100 |
| Propylene glycol | 3.00 | 3.00 |
| Acrylate/C10-30 Alkylacrylate Crosspolymer | 0.60 | 0.60 |
| NaOH 30% | 0.40 | 0.40 |
| Alcohol | 5.00 | 5.00 |
| Disodium EDTA | 0.10 | 0.10 |
| Sodium ascorbyl phosphate | 0.30 | 0.30 |
| D-Panthenol | 1.00 | 1.00 |
| Squalane | 2.00 | 2.00 |
| Coco-Carylat/Caprat | 4.00 | 4.00 |
| BHT | 0.05 | 0.05 |
| Phenoxyethanol & Methylparaben & Ethylparaben & | 0.80 | 0.80 |
| Propylparaben & Butylparaben & Isopropylparaben | | |
| Cyclomethicon | 4.00 | 4.00 |
| Glycerol | 3.00 | 3.00 |
| Tocopherylacetate | 0.50 | 0.50 |
| Phytanic acid | 0.50 | 1.00 |
| Triethanolamine | q.s. | q.s |
| | Formulation | |
| | examples | |
| | 11 | 12 |
| | % | % |
| Ingredients | (w/w) | (w/w) |
| Aqua | Ad. 100 | Ad. 100 |
| Butylene glycol | 4.00 | 4.00 |
| Acrylate/C10-30 Alkylacrylate Crosspolymer | 0.60 | 0.60 |
| NaOH 30% | 0.40 | 0.40 |
| Cyclomethicon | 5.00 | 5.00 |
| Disodium EDTA | 0.10 | 0.10 |
| D-Panthenol | 0.50 | 0.50 |
| Phenoxyethanol & Methylparaben & Ethylparaben & | 0.80 | 0.80 |
| Propylparaben & Butylparaben & Isopropylparaben | | |
| Glycerol | 3.00 | 3.00 |
| Polysorbate 20 | 0.80 | 0.80 |
| Phytonadion (Vitamin K1) | 0.10 | 0.10 |
| Tocopherylacetate | 0.10 | 0.10 |
| Phytanic acid | 0.50 | 1.00 |

Formulation examples 5 and 6 are face creams having an anti-wrinkle effect, formulation examples 7 and 8 are creams for sensitive skin, formulation examples 9 and 10 represent a skin protection body lotion, and formulation examples 11 and 12 are an eye contour gel.

The effectiveness of the formulations according to the invention can be checked by applying topically to test persons suffering from cellulite and/or subcutaneous fat pads a suitable amount of formulation 1 or 2, for example. The application is made e.g. in an amount of 20 mg of the formulation from formulation example 2 per 10 cm² skin, e.g. three times a day. After a suitable treatment period, e.g. after 2 months, the test persons show a clearly visible improvement of the cellulite.

1. Preparation for the topical administration of a compound of formula 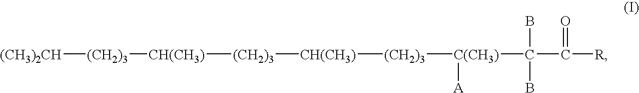
wherein R is hydrogen, OR¹, N(OH)R¹ or NR²R³,
R¹, R² and R³ are independently hydrogen, C₁-C₂₂ alkyl, C₁-C₂₂ alkenyl, C₇-C₁₂ arylalkyl (in particular benzyl, phenethyl and phenylpropyl), retinyl, tocopheryl, ascorbyl or a residue derived from an amino acid or peptide, and A and B represent hydrogen atoms or A and a residue B form a double bond and the other residue B represents a hydrogen atom or residue A is a hydrogen atom and residues B jointly form an oxygen atom or one of residues B is a hydroxyl group and the other residue B and residue A are hydrogen atoms, containing a compound of formula (I) and a pharmaceutically and/or cosmetically compatible carrier, with the proviso that the preparation contains no retinoid.

2. Preparation according to claim 1, characterized in that the compound of formula I is a compound of formula II 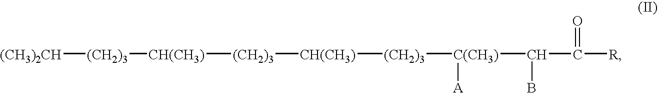
wherein R is hydrogen, OR¹ or NHR¹, N(OH)R¹,
R¹ is hydrogen, C₁-C₂₂ alkyl, C₁-C₂₂ alkenyl, benzyl, phenetyl, phenpropyl, retinyl, tocopheryl, ascorbyl or a residue derived from an amino acid or a peptide and A and B are either both hydrogen atoms or jointly form a double bond.

3. Preparation according to claim 2, characterized in that R is a hydrogen atom or a residue OR¹, R¹ is a hydrogen atom or a C₁-C₈ alkyl residue and A and B are both hydrogen atoms.

4. Preparation according to claim 1, characterized in that phytanic acid is concerned.

5. Preparation according to claim 1, characterized in that the preparation is a cosmetic preparation and the carrier is a cosmetically compatible carrier.

6. Preparation according to claim 1, characterized in that the preparation contains another active substance, selected from caffeine, flavones and isoflavones.

7. Preparation according to claim 1, characterized in that a hair care product is concerned.

8. Preparation according to claim 7, characterized in that a shampoo or a deep conditioner is concerned.

9. Preparation according to claim 1, characterized in that the preparation is a medicament and the carrier is a pharmaceutically compatible carrier.

10. Use of a compound of formula (I) as defined in claim 1, for the production of a medicament or cosmetic preparation to be administered topically to prevent and/or treat cellulite, subcutaneous fat pads, skin aging, conditions caused by a damaged or injured skin barrier, for treating hair and scalp, for treating and actively preventing dry skin and for strengthening the barrier function of the skin as well as for treating, caring for and preventing sensitive skin and/or for treating and preventing the symptoms of a negative change in the physiological homeostasis of the healthy skin, in particular inadequate, sensitive or hypoactive skin conditions or inadequate, sensitive or hypoactive conditions of skin appendages, inflammatory skin conditions as well as the atopic eczema, the polymorphous photodermatosis, psoriasis, vitiligo, sensitive, itching or irritated skin, changes in the normal lipid peroxidation, a change in the ceramide, lipid und energy metabolisms of the healthy skin, a change in the physiological transepidermal water loss, a reduction of the skin hydration and decrease of the moisture content of the skin, a change in the natural moisturizing factor content, a reduction of the cell-to-cell communication, deficiency symptoms of the intracellular DNA synthesis, DNA damage and reduction of endogenous DNA repair mechanisms, activation of metalloproteinases and/or other proteases or inhibition of the corresponding endogenous DNA repair mechanisms and deviations from the normal post-translational modification of connective tissue constituents.

11. Use according to claim 10, wherein the medicament or cosmetic preparation is a preparation for treating or preventing cellulite and/or subcutaneous fat pads.

12. Use according to claim 10, wherein the medicament or the cosmetic preparation is a preparation for treating or preventing greasy hair and/or the formation of dandruff.

13. Use of a compound of formula (I) as defined in claim 1, for treating and preventing cellulite, subcutaneous fat pads, skin aging, conditions caused by a damaged or injured skin barrier, for treating hair and scalp, for treating and actively preventing dry skin and for strengthening the barrier function of the skin, and for treating, caring for and preventing sensitive skin and/or for treating and preventing the symptoms of a negative change in the physiological homeostasis of the healthy skin, in particular inadequate, sensitive or hypoactive skin conditions or inadequate, sensitive or hypoactive conditions of skin appendages, inflammatory skin conditions and the atopical eczema, of polymorphous photodermatosis, psoriasis, vitiligo, sensitive, itching or irritated skin, changes in the normal lipid peroxidation, a change in the ceramide, lipid and energy metabolism of the healthy skin, a change in the physiological transepidermal water loss, a reduction of the skin hydration and decrease of the moisture content of the skin, a change in the natural moisturizing factor content, a reduction of the cell-to-cell communication, deficiency symptoms of the intracellular DNA synthesis, DNA damage and reduction of endogenous DNA repair mechanisms, activation of metalloproteinases and/or other proteases or inhibition of the corresponding endogenous DNA repair mechanisms and deviations of the normal post-translational modifications of connective tissue components, the treatments being cosmetic treatments.

14. Use according to claim 13 for treating and preventing cellulite and/or subcutaneous fat pads.

15. Use according to claim 13 for treating and preventing greasy hair and/or dandruff formation.

16. Compound of formula (I) as defined in claim 1, wherein residue R represents OR¹ and R¹ is an n-propyl or a C₄-C₂₂ alkyl residue.
